(19) United States
(12) Patent Application Publication
(10) Pub. No.: US 2017/0258943 A1
(43) Pub. Date: Sep. 14, 2017

(54) Using Heavy Water as a Contrast Agent for Hydrogen Magnetic Resonance Imaging

(71) Applicant: National Tsing Hua University, Hsinchu City (TW)

(72) Inventor: Fu-Nien Wang, Hsinchu City (TW)

(21) Appl. No.: 15/591,316

(22) Filed: May 10, 2017

Related U.S. Application Data
(63) Continuation-in-part of application No. 14/556,329, filed on Dec. 1, 2014.

(51) Int. Cl. A61K 49/06

(52) U.S. Cl. CPC A61K 49/06

(57) ABSTRACT

A method of using an imaging contrast agent is provided for hydrogen magnetic resonance imaging (H MRI). The agent uses replacement and chemical exchange of hydrogen (H) and deuterium (D) on obtaining MRI images for comparison. An isotonic physiologic saline solution with deuterium oxide (D₂O) is made. The solution is intravenously injected to obtain the intensity alterations on MRI images. The injected D₂O is perfused into tissue and replaces the original water. Exchanges between H and D occur and a solution of hydrogen deuterium oxide (HDO) is obtained. After such mechanisms, MRI images are compared for differences. Thus, a novel, non-radioactive, non-toxic and non-invasive MRI agent is provided for people who are allergic to general imaging agents.

### CROSS-REFERENCE TO RELATED APPLICATIONS

This application is a continuation-in-part of U.S. application Ser. No. 14/556,329, filed Dec. 1, 2014 (7000.637.2), which is a continuation-in-part of U.S. patent application Ser. No. 13/555,377, filed Jul. 23, 2012 (7000.637), a non-provisional of provisional application 61/567,246, filed Dec. 6, 2011, the disclosures of which are incorporated by reference in their entirety.

### TECHNICAL FIELD OF THE INVENTION

The present invention relates to an imaging contrast agent; more particularly, relates to using replacement effect and chemical exchange effect of heavy water (deuterium oxide, D₂O) in normal water (H₂O) as a mechanism for dynamic magnetic resonance imaging (MRI) detection by comparing images before and after administration of D₂O.

### DESCRIPTION OF THE RELATED ARTS

Cerebral blood flow (CBF) is a specific flow amount of arterial blood to the microvascular bed in a tissue, which is usually expressed in units of ml/(100 g-min). CBF has deep influence on the physiological environment, including supplement of glucose and adjustment of pH value. In an MRI image, a gadolinium(Gd)-containing imaging agent (Gd-DTPA) is most often used for obtaining data of cerebral blood perfusion. However, the shortcomings of Gd-DTPA should be taken into account. Gd-DTPA is an intravascular tracer which is more suitable for cerebral blood volume (CBV) measurement. On the other hand, there is an allergy problem to certain patients. It is revealed by James Varani, Marissa DaSilva, et al in “Effects of gadolinium-based magnetic resonance imaging agents on human skin in organ culture and human skin fibroblasts,” Inves Radio, 44: 74-81, 2009, that GD-DTPA is possible to cause nephrogenic systemic fibrosis (NSF) to patients having Kidney diseases.

Deuterium (D) is a stable isotope of hydrogen (H). As is revealed in “Deuterium NMR tissue perfusion measurements using the tracer uptake approach: II. Comparison with microspheres in tumors,” Magn Reson Med 42: 240-247, 1999, by Nicholas E. Simpson and Jeffrey L. Evelhoch, heavy water is often used in MRI as a diffusible tracer for perfusion measurement under a larmor frequency of 6.53 MHz/T. But, owing to different coils, information thus obtained in MRI has low signal-to-noise ratio (SNR).

In addition, it is also revealed in “Arterial spin labeling perfusion MRI in pediatric arterial ischemic stroke: initial experiences,” J Magn Reson Imag 29: 283-290, 2009, by Juan Chen, Daniel J. Licht, et al, that, for patients who are allergic to Gd-DTPA, arterial spin labeling (ASL) is another choice for perfusion MRI. Therein, arterial blood is used as an endogenous tracer and ASL becomes a safe and non-invasive method for CBF. Yet, the limitation for ASL is low signal change. Under an optimal tracer labeling, the signal change is only achieved at the level of 4%.

Hence, the prior arts do not fulfill all of the users' requests on actual use.

### SUMMARY OF THE INVENTION

The main purpose of the present invention is to use the replacement effect and chemical exchange effect of D₂O as a contrast mechanism for hydrogen (H) MRI. Blood flow can be assessed by dynamic MRI detection and comparing images before and after the administration of D₂O. Measurement of Deuterium can be an indirect measurement.

The second purpose of the present invention is to provide a fast, non-radioactive, non-toxic and non-invasive agent used in perfusion MRI for patients who are unsuitable for general imaging agents.

The third purpose of the present invention is to make an isotonic physiologic saline solution containing heavy water as contrast agent for H MRI. The H signal properties, including proton density, T1, and T2, can be changed and thus generated image contrast.

To achieve the above purposes, the present invention is a contrast agent of D₂O for H MRI. By administration of D₂O into tissue, part of the H₂O can be replaced by D₂O and therefore reduced the proton density property of H MRI. The D₂O and H₂O have chemical exchange effect and generate HDO. The HDO slows the T1 and T2 relaxation rates of H MRI. By affecting the proton density, T1, and T2, the signal intensities of H MRI can be altered. Accordingly, a novel agent using replacement effect and chemical exchange effect of heavy water for H MRI is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1A is the view showing the T1 relaxation rate R1 in concentration phantom;

FIG. 1B is the view showing the T2 relaxation rate R2 in concentration phantom;

FIG. 2A is the view showing the consecutive MRI images;

FIG. 2B is the view showing the curve of signal change;

FIG. 3A is the view showing the brain image before injecting D₂O;

FIG. 3B is the view showing the brain image after injecting D₂O; and

FIG. 4 is the view showing the curves of signal change of D₂O and Gd-DTPA.

FIG. 5 shows a.TSE (Turbo Spin Echo) images with tumor ROI (region of interest) shown as a red line plotted according to heterogeneity. Row b and row c represent the Ktrans parametric maps of D₂O and Gd-DTPA, respectively for each tumor ROI.

FIG. 6 shows the relative concentration time courses of TSE images of three tumors in mouse brains.

### DESCRIPTION OF THE PREFERRED EMBODIMENT

The following description of the preferred embodiment is provided to understand the features and the structures of the present invention.

The present invention is a contrast agent of heavy water (D₂O) for hydrogen magnetic resonance imaging (H MRI), where H MRI indirectly detects the deuterium (D) by measuring the difference of image intensities of H1 before and after administration of D₂O. According to the theory of nuclear magnetic resonance, the signal-to-noise ratio (SNR) is improved by indirect detection since the H sensitivity is 100 times higher than the D.

Magnetic resonance imaging (MRI) is a method in which a magnetic field sent from the MRI machine aligns the hydrogen protons in the patient's body along the same vector. The radio waves then knock the particles out of the aligned positions. As the nuclei realign into proper position, the nuclei send out radio signals. These signals are received by a computer, analyzed and converted into an image.

Any nucleus with a net nuclear spin could potentially be imaged with MRI. Such nuclei include helium-3, lithium-7, carbon-13, fluorine-19, oxygen-17 and sodium-23. Previously, when D₂O was used as an imaging agent, the D was imaged directly.

The present invention uses D₂O as a blood tracer for H MRI perfusion imaging. A solution of D₂O is intravenously injected into a living tissue or organ during consecutive H MRI acquisitions. D₂O is injected at an amount of 0.1˜20% of weight of the living object. Deuterium oxide (²H₂O or D₂O) contains a larger amount of the hydrogen isotope deuterium (D or 2H) rather than the common hydrogen-1 (¹H) isotope that makes up most of the hydrogen in normal water. The present invention identified that if an indirect method was used, the injected deuterium (²H) can be indirectly measured by measuring the signal of ¹H. The advantage of indirect detection, according to Boltzmann equilibrium law, is that the signal strength of ¹H is up to about 100 times that of ²H (see Wang, et al “Water signal attenuation by D₂O infusion as a novel contrast mechanism for ¹H perfusion MRI” NMR Biomed. 2013; 26: 692-698, the contents of which are incorporated by reference in their entirety.)

After injection of D₂O, the D₂O will be perfused to living tissue or organ by blood perfusion, and thus the image intensity of H MRI will be changed. There are two mechanisms to induce signal alteration. First, the blood perfusion will replace the original H with D. The total amount of H in imaging voxels will be reduced by this replacement effect. The decreased density of H will reduce the signal intensities of H MRI. Second, a chemical exchange phenomenon will occur between H and D and slow the T1 and T2 relaxation of H. Due to similar physical and chemical properties of D and H, the introduction of D₂O into H₂O results in an isotopic H-D exchange and leads to a production of semi-heavy water HDO by the following reaction:

$${H}_{2} O + {D}_{2} O ⇄ 2 HDO K = \frac{{[ HDO ]}^{2}}{[ {H}_{2} O ] [ {D}_{2} O ]}$$

Therein, K is an equilibrium constant experimentally determined by NMR, mass spectroscopy, and near-infrared spectroscopy and is approximate to 4 in liquid phase. The H on the HDO has slower T1 and T2 relaxation than original HDO. The averaged T1 and T2 relaxation rate are therefore decreased and further alter the image intensities. The chemical exchange of ¹H and ²H is spontaneous. The advantage of indirect detection is that the extent of ¹H-²H exchange can be detected by measuring the T1 and T2 relaxation rates of ¹H (Wang et al.). The relaxation rates are decreased when ¹H-²H exchanges occur. The measuring procedure for relaxation rates for discovering the ¹H-²H exchange after using deuterium oxide as a contrast agent is a new application.

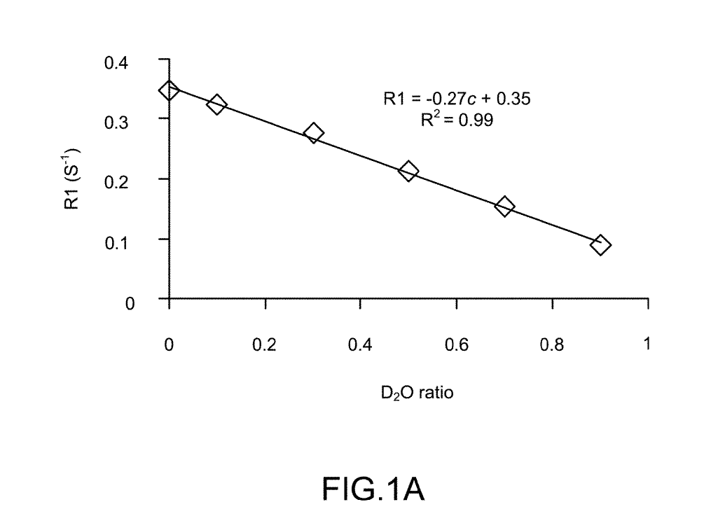

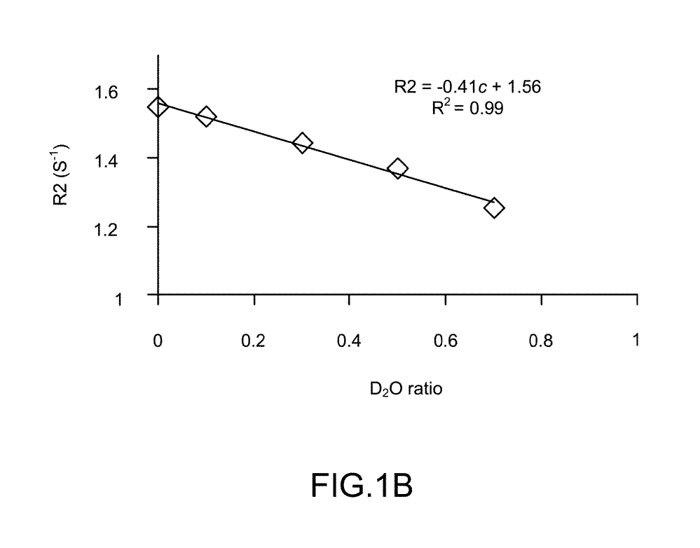

Please refer to FIG. 1A and FIG. 1B, which are views showing the T1 and T2 relaxation rates of H are linearly decreased with the D₂O concentration. The R1 and R2 in the vertical axis are the relaxation rates of T1 and T2 relaxation, respectively. The negative slopes of these two figures show that the present invention induces negative relaxivities for 1H MRI.

As discussed in Wang et al., based on the presently claimed invention, the method uses indirect measurement to enhance sensitivity (up to 100 fold). When D₂O is administered, the molecules enter the bloodstream and perfuse into the tissue from the capillary bed. The ¹H in the tissue is partially replaced by deuterium. As a result, the proton density for ¹H MRI is attenuated by the replacement effect of isotopes. Therefore, the negative contrast of conventional ¹H MRI can be induced. As the D₂O tracer is detected indirectly by ¹H with a higher gyromagnetic ratio, the sensitivity could be enhanced 100 times more strongly than is possible by the direct detection of deuterium.

In an experimental phantom with D₂O concentration as c, respective concentrations of H₂O, HDO, and D₂O are (1-c)², 2c(1-c), and c², where a part of c of H is replaced by D. Population ratio of H coupled H and D coupled H is calculated as (1-c):c. The observed T1 and T2 relaxation rates are the population weighted sums of all H. Since the D coupled H has slower relaxation rates than H coupled H, we observe linear relations of R1 and R2 as changing the D₂O concentration c in FIG. 1A and FIG. 1B.

For application of the present invention for perfusion assessment, an isotonic physiologic saline solution containing D₂O is made to be intravenously injected into a living object for H MRI.

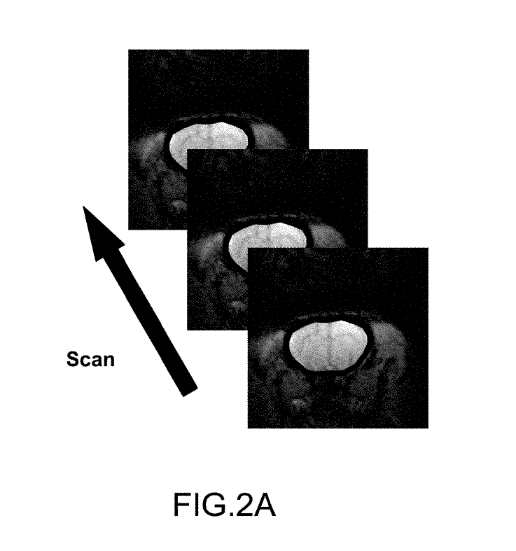

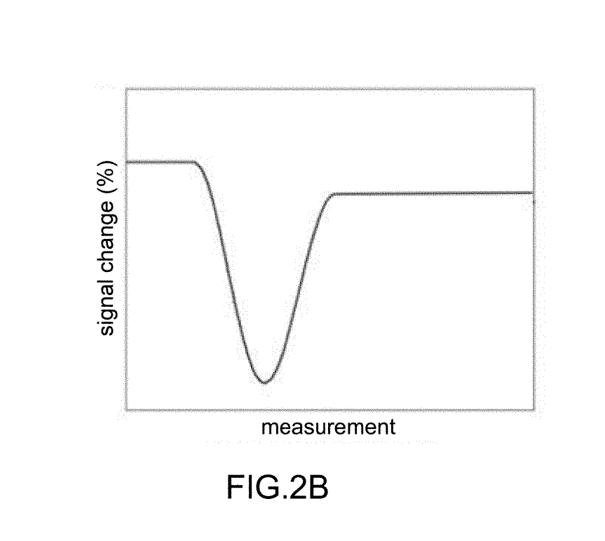

Please refer to FIG. 2A and FIG. 2B, which are views showing consecutive MRI images and a curve of signal change. As shown in the figures, 6 normal adult Sprague-Dawley rats (˜200-310 g) are used. Each rat is anesthetized with 1.5% isoflurane/air via a nose cone with respiratory monitoring. Tail vein catheterization is performed with a 0.8-m long polyethylene tube connected to a 23-gaude needle, where a dead volume in a catheter is about 0.2 ml. On operating, 1.5 mL of D₂O (99.8%, Cambridge Isotope, Woburn, Mass.) is manually injected into tail vein. After injecting, the catheter is flushed with 0.5 mL 0.9% NaCl solution. For comparison, an additional 0.2 mL Gd-DTPA (Magnevist) is manually injected into tail vein while D₂O scanning is completed after 10 min. After injection, the catheter is flushed with 0.5 mL 0.9% NaCl solution. In vivo brain imaging is performed in prone position. All images are acquired on a 4.7 animal MRI scanner (Bruker Biospec 47/40). The parameters for the dynamic images are as follow: TR/TE/θ=1000 ms/30 ms/90 degree, FOV=2.9 cm, matrix size=128×128, and slice thickness=1 mm.

Averaged signals from whole brain are portrayed as a signal-intensity curve. For comparison, the signal-intensity curve is transferred into a percentage change curve according to the following formula:

$$signal change ( \% ) = \frac{S ( t ) - {S}_{0}}{{S}_{0}} \times 100 \%$$

Therein, S(t) is a signal at time t and S0 is a signal before using the contrast agent. Expression of data analysis is shown in FIG. 2A and FIG. 2B. After manually selecting the whole brain as ROI in FIG. 2A, the dynamic points are plotted as signal intensity curves and then transferred into the signal change curves as shown in FIG. 2B.

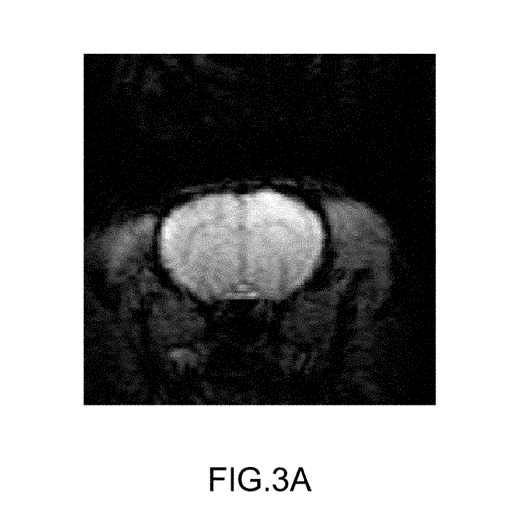

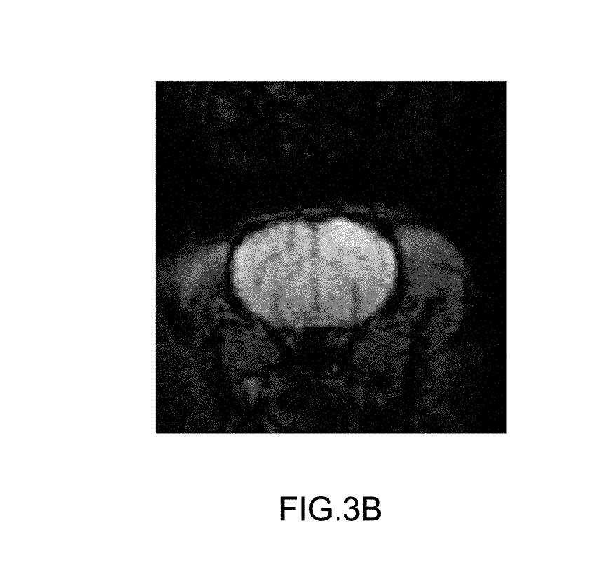

Please refer to FIG. 3A and FIG. 3B, which are views showing brain images before and after injecting D₂O. As shown in the figures, after D₂O injection, original H₂O are replaced by D₂O. Because of the replacement effect and chemical exchange effect, the signal intensities decreased as shown in FIG. 3A to FIG. 3B.

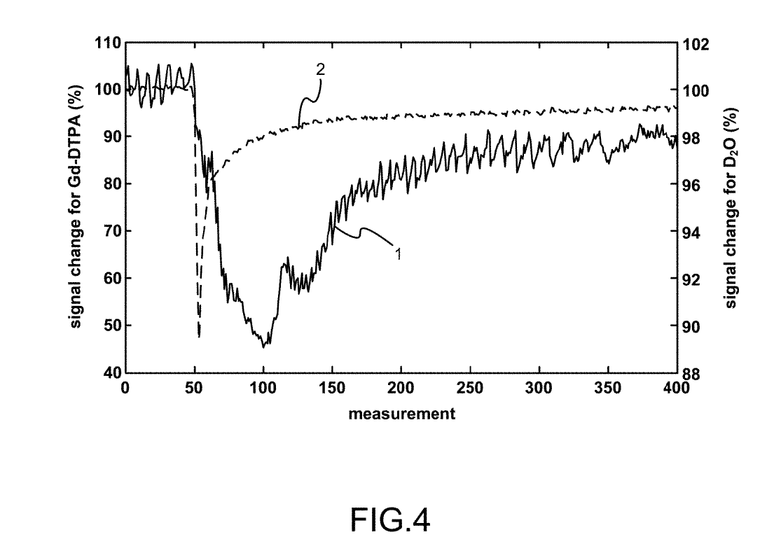

Please refer to FIG. 4, which is a view showing curves of signal change of D₂O and Gd-DTPA. As shown in the figure, a curve of signal change for D₂O 1 and a curve of signal change for Gd-DTPA 2 are displayed. Although the signal change for D₂O injection is not as strong as that of Gd-DTPA, the signal change of 10% is good enough for the measurement of tissue perfusion.

D₂O is not toxic to animals as long as the amount of injecting D₂O is less than 20% of body weight. The injected D₂O is about 0.6% of body weight in the present invention. It is much lower than the tolerable dosage. From the results, the signal change of D₂O achieves at the level of 10%. It is more superior to that of arterial spin labeling (ASL).

### Use of D₂O for Imaging a Mouse Brain Tumor

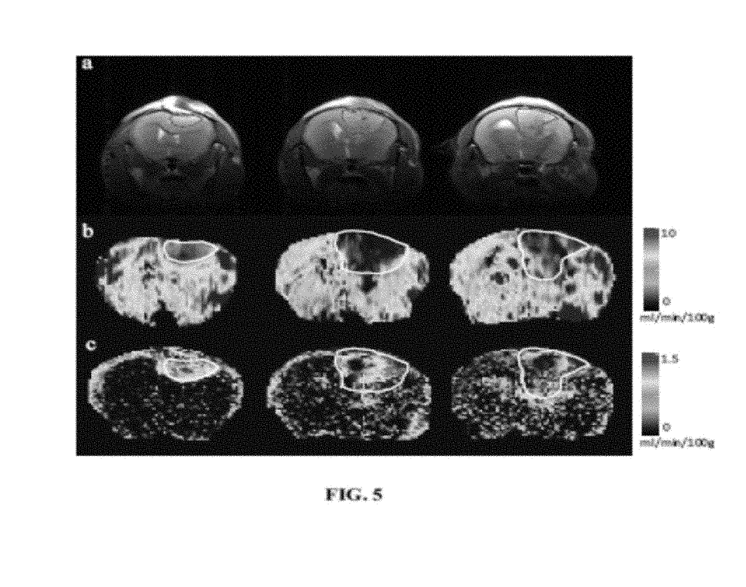

FIG. 5 shows the results of a study comparing the use of D₂O and Gd-DTPA as contrast agents for perfusion MRI of a mouse tumor model. The most commonly used contrast agents in the clinic are Gd-based chelates. Because deuterium oxide (D₂O) is an alternative contrast agent, we decided to compare D₂O as a contrast agent for rodent brain perfusion by monitoring the attenuation of the ¹H signal and to compare the results to those with Gd-DPTA on the same sample. The indirect detection of D₂O achieved better SNR and image resolution than previously used direct detection. Since D₂O is a highly diffusible contrast agent, the results provided by Gd chelates and D₂O are different. In the literature, the D₂O has been utilized to observe the tumor perfusion on a mouse model by direct detection, but in this study, we aimed to re-investigate the perfusion information carried by D₂O with advanced spatial resolution indirectly.

ALTS1C1 tumor cells were prepared by cell culture in medium containing 10% fetal bovine serum (FBS), which would grow glioma in mouse brain. 105 cells/mL of ALTS1C1 cell (˜2 μL) were intracerebrally inoculated into C57BL/6J mice (N=5, weight: 18˜30 g). After fourteen days of tumor growth, MRI scanning was performed. All procedures complied with the norms of animal experiments. Mice were scanned under 3% isoflurane anesthesia with oxygen on a 7T Bruker Clinscan scanner. Isotonic D₂O contained 0.9% w/w NaCl and a 2 mL/100 g dose was administered through the tail vein within 20 s with a syringe pump. Using a saline solution makes it isotonic to tissue. Turbo spin-echo (TSE) was used to dynamically scan. Imaging parameters were: TR/TE=1000/14 ms, turbo factor=8, matrix size=128*64, FOV=20 mm, 6 slices with thickness=1 mm, measurement=120, temporal resolution=9 s. The relative concentration of D₂O could be calculated as: Where S0 is the average signal of 20 baseline measurements before D₂O injection of single pixel. After using D₂O as contrast agent, Gd-DTPA with 0.2 mmol/Kg dose was subsequently implemented at the same perfusion slice position and scanned with 2D-FLAIR under the assumption that the subsequent experiment was unaffected by D₂O. Imaging parameters were: TRITE=4.9/1.97 ms, FA=20, matrix size=128*128, FOV=20 mm, 3 slices with thickness=1 mm, measurement=160, temporal resolution=2.2 s. Data processing were performed by MATLAB and image were analyzed with several different DCE-MRI quantitative models to calculate the kinetic parameters which can provide perfusion information.

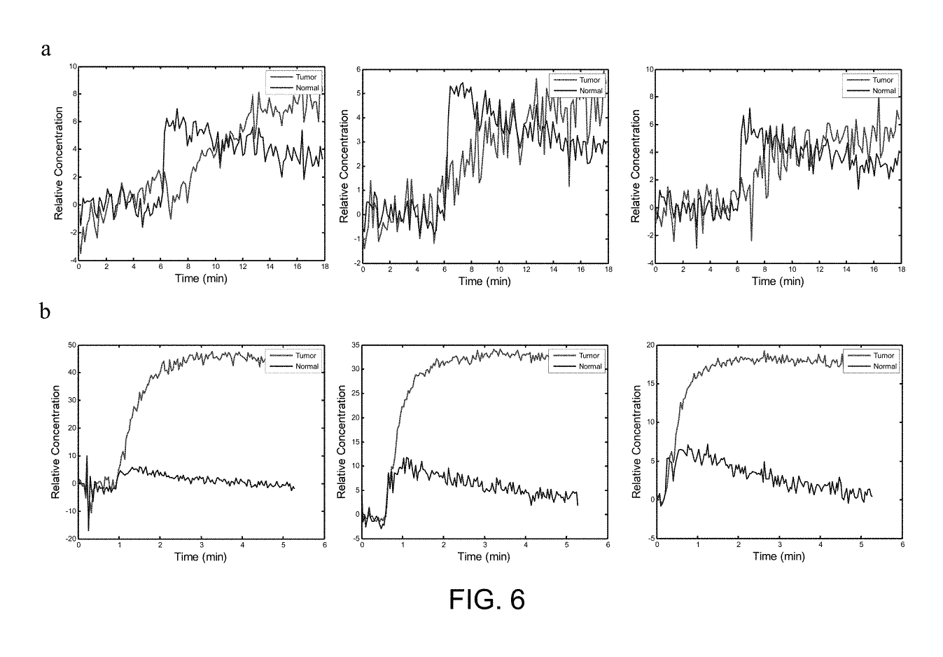

In FIG. 5, three slices of a Turbo Spin Echo (TSE) image of glioma mouse models were analyzed and the tumor region of interest (ROI) was plotted according to the heterogeneity of intensities (see red line) in FIG. 5a. A comparison was made of Ktrans parametric maps of D₂O and Gd-DTPA in FIGS. 5b and 5c, respectively. Both maps were generated from a Toft's model, and the same tumor ROI. Theoretically, the Ktrans can provide mixed information of vascular permeability and flow. In the Ktrans maps of D₂O, the Ktrans values were heterogeneously distributed and were lower inside the tumor than in the normal brain tissue (making the tumor look like a photographic negative image). The map using D₂O was different from the Ktrans maps of Gd-DTPA because the Ktrans maps of Gd-DTPA were higher inside the tumor in some hot regions than in the normal brain tissue. FIG. 6's row a and row b show the relative concentration time courses of D₂O and Gd-DTPA, respectively. Red lines represent the mean concentration curve of tumor ROI, and blue lines represent the mean concentration curve of the contralateral (nontumor) region. Note that the curve of D₂O in the normal region concentration curves have a steep wash-in slope, and then gradually decline. However, the tumor concentration curves are slowly increasing until they reach a steady value.

In DCE-MRI with a Gd-based contrast agent, the tracer is perfused into the limited extracellular-extravascular space. Gd-DTPA is only leaky in lesion tissues in brain tissue. Theoretically, the Ktrans is determined by the permeability, surface area of microvessels, and flow. In theory, due to a freely diffusible property, the Ktrans value of D₂O may represent the flow property rather than the vascular permeability or leakage condition. In our result, the tumor Ktrans of Gd-DTPA is higher than normal tissue. This result revealed the high permeability of the immature neovascularization in tumor. However, the Ktrans maps of D₂O showed a lower tumor Ktrans value. The D₂O seems more easily able to reach the normal tissue than to reach the tumor region. We speculated that it is because of the high osmotic pressure of tumor edema. Therefore, D₂O slowly diffused into tumor area and continuously exchanged with tissue water until a balanced concentration was achieved. The osmotic gradient may play a significant role when using D₂O perfusion as a diffusible tracer. Inside the tumor region, the heterogeneity shown by D₂O and Gd-DTPA are somewhat different. The data on brain tumor in a mouse model shows the feasibility of tumor diagnosis in the brain using this method. The TSE method used was able to get images with minimal distortion in a short time with low signal to noise ratio (SNR).

### Results:

Thus, the present invention uses a non-toxic and diffusible imaging agent, D₂O, to be injected into an animal (e.g. rat) for obtaining cerebral blood flow (CBF). In addition, D₂O is a potential contrast agent in perfusion MRI for patients who are unsuitable for Gd-DTPA. The data in FIGS. 5 and 6 show that the method works to image brain tumors. Accordingly, the present invention provides a fast, non-radioactive, non-toxic and non-invasive agent for MRI.

To sum up, the present invention is an agent using heavy water for hydrogen magnetic resonance imaging, where replacement effect and chemical exchange effect of D₂O are used as mechanisms for image contrast; D₂O is a potential contrast agent in perfusion MRI for patients who are unsuitable for Gd-DTPA; and, thus, the present invention provides a fast, non-radioactive, non-toxic and non-invasive agent for MRI.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of providing a fast, non-radioactive, non-toxic and non-invasive imaging contrast agent for use in perfusion MRI, comprising:
administering heavy water (deuterium oxide, D₂O) as a contrast agent of hydrogen magnetic resonance imaging (MRI); and
indirectly detecting the deuterium (D) by measuring the difference of signal intensities of hydrogen (¹H) before and after administration of D₂O, wherein the sensitivity of indirect detection of D₂O is up to 100 times higher than the direct detection.

2. The method according to claim 1, wherein said imaging contrast agent is produced by the method comprising:
(a) preparing isotonic physiologic saline by sodium chloride and D₂O;
(b) putting said solution of D₂O into a living object through intravenous injection;
(c) scanning said living object through MRI; and
(d) obtaining changes of brightness and contrast in images of said living object.

3. The method according to claim 2, wherein, on obtaining said changes of brightness and contrast, two mechanisms occur; the replacement of H with injected D, and a chemical exchange reaction between D₂O and H₂O as follows:
$${H}_{2} O + {D}_{2} O ⇄ 2 HDO , K = \frac{{[ HDO ]}^{2}}{[ {H}_{2} O ] [ {D}_{2} O ]} ;$$
wherein K is an equilibrium constant.

4. The method of claim 2, wherein the D₂O is injected into said living object at an amount of 0.1˜20% of weight of said living object.

5. The method of claim 2, wherein the D₂O is injected into said living object in a 2 ml/100 g dose in a solution of 9% w/w NaCl.

6. The method of claim 2, wherein the imaging parameters are TR/TE=1000/14 ms, turbo factor=8, matrix size=128×64, FOV=20 mm, and temporal resolution=9 s.

7. The method of claim 1, further comprising using Turbo Spin Echo for imaging.

8. The method of claim 1, wherein the concentration of D₂O is 99.8% in weight in a saline solution.

9. The method of claim 1, wherein the D₂O is added at between about 1.5 and about 3% of the body weight of the living object.

10. The method of claim 9, wherein the D₂O is added at about 2% of the body weight of the living object.

11. The method of claim 8, wherein the saline solution is 0.9% NaCl.

12. The method of claim 1, wherein the imaging uses Toft's model for a pixel by pixel analysis of the image.
